(19) United States
(12) Patent Application Publication
(10) Pub. No.: US 2024/0431200 A1
(43) Pub. Date: Dec. 26, 2024

(54) MATERIALS FOR OPTOELECTRONIC APPLICATIONS

(71) Applicant: The Regents of the University of Michigan, Ann Arbor, MI (US)

(72) Inventors: Stephen R. Forrest, Ann Arbor, MI (US); Yongxi Li, Ann Arbor, MI (US)

(21) Appl. No.: 18/737,742

(22) Filed: Jun. 7, 2024

Related U.S. Application Data
(60) Provisional application No. 63/508,408, filed on Jun. 15, 2023.

(51) Int. Cl. H10K 85/60; C07D 249/18; C07D 285/14; C07D 417/14; C07D 519/00; H10K 30/30

(52) U.S. Cl. CPC H10K 85/657; C07D 249/18; C07D 285/14; C07D 417/14; C07D 519/00; H10K 85/6572; H10K 30/30

(57) ABSTRACT

Provided are compounds of Formula (I). Also provided are formulations comprising these compounds. Further provided are optoelectronic devices that utilize these compounds.

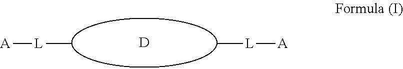

### CROSS-REFERENCE TO RELATED APPLICATIONS

The present application claims priority to U.S. Provisional Application No. 63/508,408, filed Jun. 15, 2023, which is incorporated by reference herein in its entirety.

### BACKGROUND

Optoelectronic devices rely on the optical and electronic properties of materials to either produce or detect electromagnetic radiation electronically or to generate electricity from ambient electromagnetic radiation. Opto-electronic devices that make use of organic materials are becoming increasingly desirable for a number of reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting diodes/devices (OLEDs), organic phototransistors, organic photovoltaic cells, and organic photodetectors.

Organic photovoltaic cells (OPVs) have sparked considerable interest in recent years owing to their flexibility, light weight, non-toxic nature and semi-transparency that makes them ideal for building integrated and building applied applications. Compared to the commercial solar modules that typically have power conversion efficiencies of PCE=15-22% of sunlight, organics have improved from 10% in 2016 to over 17% in 2019 for single junction devices. Advances have steadily continued, headed toward single junction thermodynamically limited efficiencies of ˜25%, with potentially higher efficiencies based on multijunction cells. This rapid advance has been paced by the development of non-fullerene acceptors (NFAs).

An essential feature of all high-performance photovoltaic devices that exhibit an acceptable level of reproducibility and a long operational lifetime, is that the materials of which are they comprised are of high purity. In crystalline Si photovoltaic cells, the chemical purity of Si has been an important limiting factor in their performance. The introduction of extrinsic impurities can result in significant changes in conductivity by creating lattice strain or by forming interstitial and substitutional defects. As a consequence, the precise control of source material purity is a common strategy for improving the efficiency and reliability of photovoltaics.

In contrast to inorganic photovoltaics, understanding the effects of impurities on organic photovoltaics (OPVs) has received little attention despite their potentially profound impact on performance. In fact, the molecular contaminants left over from synthesis, or that result from decomposition during device operation, can dramatically impact the intrinsic optical and electrical properties of pristine OPV materials (Mateker, W. R. et al. Energy Environ. Sci. 6, 2529-2537 (2013); Salzman, R. F. et al. Org. Electron. 6, 242-246 (2005); Tetreault, et al., Org. Electron. 92, (2021)). One example is the emergence of photoinduced dimerized products of the fullerene acceptor, C₆₀, during OPV operation, which reduces the exciton lifetime and diffusion length, leading to a significant decrease in device lifetime (Wang, N., et al., Sol. Energy Mater. Sol. Cells 125, 170-175 (2014)). Beyond these changes, impurities can disrupt the crystalline order in molecular solids (Forrest, S. R., et al., J. Appl. Phys. 56, 543-551 (1984)), thereby reducing the charge carrier mobility and cell power conversion efficiency (PCE). Therefore, considerable care must be taken to remove contaminants from the source materials, and avoid material decomposition during device preparation and operation.

Ternary blend OPVs comprising one donor and two acceptors, or one acceptor and two donors have attracted great interest in recent years (Lu, L., et al., Nat. Photonics 8, 716-722 (2014); Gasparini, N., et al., Nat. Rev. Mater. 4, 229-242 (2019); Yu, R., et al., Adv. Energy Mater. 8, 1702814 (2018); Li, Y. et al. J. Am. Chem. Soc. 141, 18204-18210 (2019); Li, Y. et al. Adv. Mater. 30, 1804416 (2018)). Compared with conventional binary component devices, the introduction of a third molecular species into the bulk heterojunction (BHJ) layer can increase solar spectral coverage, reduce the energy loss, and significantly improve OPV efficiency. In particular, the rapid development of acceptor (A)—donor (D)—acceptor (A) non-fullerene acceptors (NFAs) has led to ternary blend OPVs comprising two NFAs and a polymer donor with PCE approaching 20% under 1 sun intensity, AM 1.5G illumination (Cui, Y. et al. Adv. Mater. 33, 2102420 (2021); Zuo, L. et al. Nat. Nanotechnol. 17, 53-60 (2022); Zhan, L. et al. Adv. Energy Mater. 12, 2201076 (2022); Hultmark, S. et al. Adv. Funct. Mater. 30, 2005462 (2020)).

### SUMMARY OF THE DISCLOSURE

In one aspect, the present disclosure relates to a compound of Formula (I):

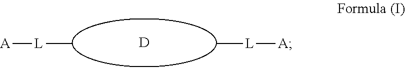

wherein:
D represents a linear ring system of between three and twenty-five rings D′; wherein each ring D′ is independently a monocyclic ring or a polycyclic fused ring system; and wherein each ring D′ is a 5-membered to 10-membered carbocyclic or heterocyclic ring; wherein each ring D′ is fused to an adjacent ring D′ or bonded to an adjacent ring D′ via a single bond; and wherein each ring D′ is optionally substituted with one or more substituents R;
each L is independently a divalent linker covalently bonded to a terminal ring D′ and to A; and
each A is independently an acceptor group selected from the group consisting of:

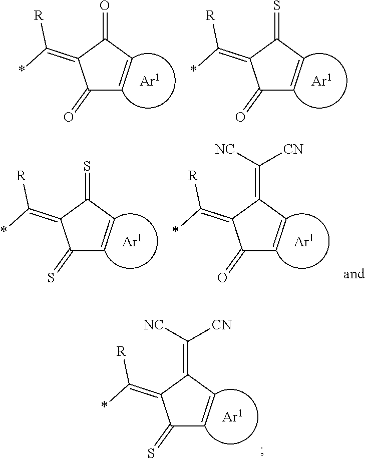

wherin each Ar¹ is independently an aromatic or heteroaromatic ring;
* represent the bond to L;
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
any two adjacent substituents R optionally join to form a fused ring.

In another aspect, the present disclosure provides a formulation comprising a compound of Formula (I) as described herein.

In yet another aspect, the present disclosure provides an optoelectronic device comprising a compound of Formula (I) as described herein.

### BRIEF DESCRIPTION OF THE DRAWINGS

For a more complete understanding of the disclosure, reference is made to the following detailed description and accompanying drawing figures, in which like reference numerals may be used to identify like elements in the figures.

FIG. 1 is a depiction of a proposed mechanism for the end-capping exchange reaction through formation of an aldehyde intermediate.

FIG. 2 depicts the effect of end-capping units on the reactivity of exchange process. At left are the chemical structures of the investigated molecules. At right is a comparison of the ¹⁹F{¹H} NMR spectra of the crude mixture of BTIC-4F and investigated molecule. The mixture was obtained by stirring in chlorobenzene at 65° C. for 24h.

### DETAILED DESCRIPTION

The present disclosure provides non-fullerene acceptors (NFAs) with good photostability and morphological stability. In some aspects, employing these NFAs in the one or more device architectures, OPV device is achieved with improved the operational lifetime.

Various non-limiting examples of OPVs and compositions within various layers of an OPV are described in greater detail below.

### Definitions

As used herein, the term “organic” includes polymeric materials as well as small molecule organic materials that may be used to fabricate organic opto-electronic devices. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety. The core moiety of a dendrimer may be a fluorescent or phosphorescent small molecule emitter. A dendrimer may be a “small molecule,” and it is believed that all dendrimers currently used in the field of optoelectronic devices are small molecules.

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processable” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

As used herein, the terms “electrode” and “contact” may refer to a layer that provides a medium for delivering current to an external circuit or providing a bias current or voltage to the device. For example, an electrode, or contact, may provide the interface between the active regions of an organic photosensitive optoelectronic device and a wire, lead, trace or other means for transporting the charge carriers to or from the external circuit. Examples of electrodes include anodes and cathodes, which may be used in a photosensitive optoelectronic device.

As used herein, the term “transparent” may refer to a material that permits at least 50% of the incident electromagnetic radiation in relevant wavelengths to be transmitted through it. In a photosensitive optoelectronic device, it may be desirable to allow the maximum amount of ambient electromagnetic radiation from the device exterior to be admitted to the photoconductive active interior region. That is, the electromagnetic radiation must reach a photoconductive layer(s), where it can be converted to electricity by photoconductive absorption. This often dictates that at least one of the electrical contacts or electrodes should be minimally absorbing and minimally reflecting of the incident electromagnetic radiation. In some cases, such a contact should be transparent or at least semi-transparent. In one embodiment, the transparent material may form at least part of an electrical contact or electrode.

As used herein, the term “semi-transparent” may refer to a material that permits some, but less than 50% transmission of ambient electromagnetic radiation in relevant wavelengths. Where a transparent or semi-transparent electrode is used, the opposing electrode may be a reflective material so that light which has passed through the cell without being absorbed is reflected back through the cell.

As used and depicted herein, a “layer” refers to a member or component of a device, for example an optoelectronic device, being principally defined by a thickness, for example in relation to other neighboring layers, and extending outward in length and width. It should be understood that the term “layer” is not necessarily limited to single layers or sheets of materials. In addition, it should be understood that the surfaces of certain layers, including the interface(s) of such layers with other material(s) or layers(s), may be imperfect, wherein said surfaces represent an interpenetrating, entangled or convoluted network with other material(s) or layer(s). Similarly, it should also be understood that a layer may be discontinuous, such that the continuity of said layer along the length and width may be disturbed or otherwise interrupted by other layer(s) or material(s).

As used herein, a “photoactive region” refers to a region of a device that absorbs electromagnetic radiation to generate excitons. Similarly, a layer is “photoactive” if it absorbs electromagnetic radiation to generate excitons. The excitons may dissociate into an electron and a hole in order to generate an electrical current.

As used herein, the term “cathode buffer” is given its ordinary meaning in the art and generally refers to a material which is disposed between a cathode and a photoactive material. Generally, a cathode buffer material aids in reducing the work function of the cathode interface. Those of ordinary skill in the art will be able to select suitable cathode buffer materials with appropriate work functions for use in the methods and devices described herein.

As used herein, the term “anode buffer” is given its ordinary meaning in the art and generally refers to a material which is disposed between a anode and a photoactive material. Generally, a anode buffer material aids in reducing the work function of the anode interface. Those of ordinary skill in the art will be able to select suitable anode buffer materials with appropriate work functions for use in the methods and devices described herein.

As used herein, the terms “donor” and “acceptor” refer to the relative positions of the highest occupied molecular orbital (“HOMO”) and lowest unoccupied molecular orbital (“LUMO”) energy levels of two contacting but different organic materials. If the LUMO energy level of one material in contact with another is lower, then that material is an acceptor. Otherwise it is a donor. It is energetically favorable, in the absence of an external bias, for electrons at a donor-acceptor junction to move into the acceptor material, and for holes to move into the donor material.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

As used herein, the term “band gap” (E_{g}) of a polymer may refer to the energy difference between the HOMO and the LUMO. The band gap is typically reported in electron volts (eV). The band gap may be measured from the UV-vis spectroscopy or cyclic voltammetry. A “low band gap” polymer may refer to a polymer with a band gap below 2 eV, e.g., the polymer absorbs light with wavelengths longer than 620 nm.

As used herein, the term “excitation binding energy” (E_{B}) may refer to the following formula: E_{B}=(M⁺+M⁻)-(M*+M), where M⁺ and M⁻ are the total energy of a positively and negatively charged molecule, respectively; M* and M are the molecular energy at the first singlet state (Si) and ground state, respectively. Excitation binding energy of acceptor or donor molecules affects the energy offset needed for efficient exciton dissociation. In certain examples, the escape yield of a hole increases as the HOMO offset increases. A decrease of exciton binding energy E_{B} for the acceptor molecule leads to an increase of hole escape yield for the same HOMO offset between donor and acceptor molecules.

As used herein, “power conversion efficiency” (PCE) (ηₚ) may be expressed as:

$${η}_{ρ} = \frac{{V}_{OC} * FF * {J}_{SC}}{{P}_{O}}$$

V_{OC} is the open circuit voltage, FF is the fill factor, J_{SC} is the short circuit current, and P_{O} is the input optical power.

As used herein, “spin coating” may refer to the process of solution depositing a layer or film of one material (i.e., the coating material) on a surface of an adjacent substrate or layer of material. The spin coating process may include applying a small amount of the coating material on the center of the substrate, which is either spinning at low speed or not spinning at all. The substrate is then rotated at high speed in order to spread the coating material by centrifugal force. Rotation is continued while the fluid spins off the edges of the substrate, until the desired thickness of the film is achieved. The applied solvent is usually volatile, and simultaneously evaporates. Therefore, the higher the angular speed of spinning, the thinner the film. The thickness of the film also depends on the viscosity and concentration of the solution and the solvent.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

The terms “halo,” “halogen,” and “halide” are used interchangeably and refer to fluorine, chlorine, bromine, and iodine.

The term “pseudohalogen” refers to polyatomic analogues of halogens, whose chemistry, resembling that of the true halogens, allows them to substitute for halogens in several classes of chemical compounds. Exemplary pseudohalogens include, but are not limited to, nitrile, cyaphide, isocyanide, cyanate, isocyanate, fulminate, thiocyanate, isothiocyanate, selenocyanate, tellurocyanate, azide, tetracarbonylcobaltate, trinitromethanide, and tricyanomethanide groups.

The term “acyl” refers to a substituted carbonyl radical (C(O)—Rₛ).

The term “ester” refers to a substituted oxycarbonyl (—O—C(O)—R, or —C(O)—O—Rₛ) radical.

The term “ether” refers to an —OR, radical.

The terms “sulfanyl” or “thio-ether” are used interchangeably and refer to a —SR, radical.

The term “sulfinyl” refers to a —S(O)—Rₛ radical.

The term “sulfonyl” refers to a —SO₂—Rₛ radical.

The term “phosphino” refers to a —P(Rₛ)₃ radical, wherein each Rₛ can be same or different.

The term “silyl” refers to a —Si(Rₛ)₃ radical, wherein each Rₛ can be same or different.

The term “boryl” refers to a —B(Rₛ)₂ radical or its Lewis adduct —B(Rs)3 radical, wherein Rs can be same or different.

In each of the above, R, can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof. Preferred R, is selected from the group consisting of alkyl, cycloalkyl, aryl, heteroaryl, and combination thereof.

The term “alkyl” refers to and includes both straight and branched chain alkyl radicals. Preferred alkyl groups are those containing from one to fifteen carbon atoms and includes methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl,and the like. Additionally, the alkyl group is optionally substituted.

The term “cycloalkyl” refers to and includes monocyclic, polycyclic, and spiro alkyl radicals. Preferred cycloalkyl groups are those containing 3 to 12 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group is optionally substituted.

The terms “heteroalkyl” or “heterocycloalkyl” refer to an alkyl or a cycloalkyl radical, respectively, having at least one carbon atom replaced by a heteroatom. Optionally the at least one heteroatom is selected from O, S, N, P, B, Si and Se, preferably, 0, S or N. Additionally, the heteroalkyl or heterocycloalkyl group is optionally substituted.

The term “alkenyl” refers to and includes both straight and branched chain alkene radicals. Alkenyl groups are essentially alkyl groups that include at least one carbon-carbon double bond in the alkyl chain. Cycloalkenyl groups are essentially cycloalkyl groups that include at least one carbon-carbon double bond in the cycloalkyl ring. The term “heteroalkenyl” as used herein refers to an alkenyl radical having at least one carbon atom replaced by a heteroatom. Optionally the at least one heteroatom is selected from O, S, N, P, B, Si, and Se, preferably, O, S, or N. Preferred alkenyl, cycloalkenyl, or heteroalkenyl groups are those containing two to fifteen carbon atoms. Additionally, the alkenyl, cycloalkenyl, or heteroalkenyl group is optionally substituted.

The term “alkynyl” refers to and includes both straight and branched chain alkyne radicals. Preferred alkynyl groups are those containing two to fifteen carbon atoms. Additionally, the alkynyl group is optionally substituted.

The terms “aralkyl” or “arylalkyl” are used interchangeably and refer to an alkyl group that is substituted with an aryl group. Additionally, the aralkyl group is optionally substituted.

The term “heterocyclic group” refers to and includes aromatic and non-aromatic cyclic radicals containing at least one heteroatom. Optionally the at least one heteroatom is selected from O, S, N, P, B, Si, and Se, preferably, O, S, or N. Hetero-aromatic cyclic radicals may be used interchangeably with heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group may be optionally substituted.

The term “aryl” refers to and includes both single-ring aromatic hydrocarbyl groups and polycyclic aromatic ring systems. The polycyclic rings may have two or more rings in which two carbons are common to two adjoining rings (the rings are “fused”) wherein at least one of the rings is an aromatic hydrocarbyl group, e.g., the other rings can be cycloalkyls, cycloalkenyls, aryl, heterocycles, and/or heteroaryls. Preferred aryl groups are those containing six to thirty carbon atoms, preferably six to twenty carbon atoms, more preferably six to twelve carbon atoms. Especially preferred is an aryl group having six carbons, ten carbons or twelve carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, fluorene, and naphthalene. Additionally, the aryl group is optionally substituted.

The term “heteroaryl” refers to and includes both single-ring aromatic groups and polycyclic aromatic ring systems that include at least one heteroatom. The heteroatoms include, but are not limited to O, S, N, P, B, Si, and Se. In many instances, O, S, or N are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group is optionally substituted.

Of the aryl and heteroaryl groups listed above, the groups of triphenylene, naphthalene, anthracene, dibenzothiophene, dibenzofuran, dibenzoselenophene, carbazole, indolocarbazole, imidazole, pyridine, pyrazine, pyrimidine, triazine, and benzimidazole, and the respective aza-analogs of each thereof are of particular interest.

The terms alkyl, cycloalkyl, heteroalkyl, heterocycloalkyl, alkenyl, cycloalkenyl, heteroalkenyl, alkynyl, aralkyl, heterocyclic group, aryl, and heteroaryl, as used herein, are independently unsubstituted, or independently substituted, with one or more general substituents.

In many instances, the general substituents are selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof.

In some instances, the preferred general substituents are selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof.

In some instances, the preferred general substituents are selected from the group consisting of deuterium, fluorine, alkyl, cycloalkyl, alkoxy, aryloxy, amino, silyl, aryl, heteroaryl, sulfanyl, and combinations thereof.

In yet other instances, the more preferred general substituents are selected from the group consisting of deuterium, fluorine, alkyl, cycloalkyl, aryl, heteroaryl, and combinations thereof.

The terms “substituted” and “substitution” refer to a substituent other than H that is bonded to the relevant position, e.g., a carbon or nitrogen. For example, when R¹ represents mono-substitution, then one R¹ must be other than H (i.e., a substitution). Similarly, when R¹ represents di-substitution, then two of R¹ must be other than H. Similarly, when R¹ represents no substitution, R¹, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The “aza” designation in the fragments described herein, i.e. aza-dibenzofuran, aza-dibenzothiophene, etc. means that one or more of the C—H groups in the respective aromatic ring can be replaced by a nitrogen atom, for example, and without any limitation, azatriphenylene encompasses both dibenzo[f,h]quinoxaline and dibenzo[f,h]quinoline. One of ordinary skill in the art can readily envision other nitrogen analogs of the aza-derivatives described above, and all such analogs are intended to be encompassed by the terms as set forth herein.

As used herein, “deuterium” refers to an isotope of hydrogen. Deuterated compounds can be readily prepared using methods known in the art. For example, U.S. Pat. No. 8,557,400, Patent Pub. No. WO 2006/095951, and U.S. Pat. Application Pub. No. US 2011/0037057, which are hereby incorporated by reference in their entireties, describe the making of deuterium-substituted organometallic complexes. Further reference is made to Ming Yan, et al., Tetrahedron 2015, 71, 1425-30 and Atzrodt et al., Angew. Chem. Int. Ed. (Reviews) 2007, 46, 7744-65, which are incorporated by reference in their entireties, describe the deuteration of the methylene hydrogens in benzyl amines and efficient pathways to replace aromatic ring hydrogens with deuterium, respectively.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In some instance, a pair of adjacent substituents can be optionally joined or fused into a ring. The preferred ring is a five, six, or seven-membered carbocyclic or heterocyclic ring, includes both instances where the portion of the ring formed by the pair of substituents is saturated and where the portion of the ring formed by the pair of substituents is unsaturated. As used herein, “adjacent” means that the two substituents involved can be on the same ring next to each other, or on two neighboring rings having the two closest available substitutable positions, such as 2, 2′ positions in a biphenyl, or 1, 8 position in a naphthalene, as long as they can form a stable fused ring system.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. Pat. No. 7,431,968, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and organic vapor jet printing (OVJP). Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution processability than those having symmetric structures, because asymmetric materials may have a lower tendency to recrystallize. Dendrimer substituents may be used to enhance the ability of small molecules to undergo solution processing.

Devices fabricated in accordance with embodiments of the present disclosure may further optionally comprise a barrier layer. One purpose of the barrier layer is to protect the electrodes and organic layers from damaging exposure to harmful species in the environment including moisture, vapor and/or gases, etc. The barrier layer may be deposited over, under or next to a substrate, an electrode, or over any other parts of a device including an edge. The barrier layer may comprise a single layer, or multiple layers. The barrier layer may be formed by various known chemical vapor deposition techniques and may include compositions having a single phase as well as compositions having multiple phases. Any suitable material or combination of materials may be used for the barrier layer. The barrier layer may incorporate an inorganic or an organic compound or both. The preferred barrier layer comprises a mixture of a polymeric material and a non-polymeric material as described in U.S. Pat. No. 7,968,146, PCT Pat. Application Nos. PCT/US2007/023098 and PCT/US2009/042829, which are herein incorporated by reference in their entireties. To be considered a “mixture”, the aforesaid polymeric and non-polymeric materials comprising the barrier layer should be deposited under the same reaction conditions and/or at the same time. The weight ratio of polymeric to non-polymeric material may be in the range of 95:5 to 5:95. The polymeric material and the non-polymeric material may be created from the same precursor material. In one example, the mixture of a polymeric material and a non-polymeric material consists essentially of polymeric silicon and inorganic silicon.

Devices fabricated in accordance with embodiments of the disclosure can be incorporated into a wide variety of electronic component modules (or units) that can be incorporated into a variety of electronic products or intermediate components.

The materials and structures described herein may have applications in devices other than organic solar cells. For example, other optoelectronic devices such as organic electroluminescent devices (OLEDs) and organic photodetectors may employ the materials and structures. More generally, organic devices, such as organic transistors, may employ the materials and structures.

According to one aspect, the present disclosure relates to a compound of Formula (I):

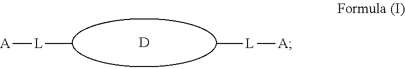

wherein:
D represents a linear ring system of between three and twenty-five rings D′; wherein each ring D′ is independently a monocyclic ring or a polycyclic fused ring system; and wherein each ring D′ is a 5-membered to 10-membered carbocyclic or heterocyclic ring; wherein each ring D′ is fused to an adjacent ring D′ or bonded to an adjacent ring D′ via a single bond; and wherein each ring D′ is optionally substituted with one or more substituents R;
each L is independently a divalent linker covalently bonded to a terminal ring D′ and to A; and
each A is independently an acceptor group selected from the group consisting of:

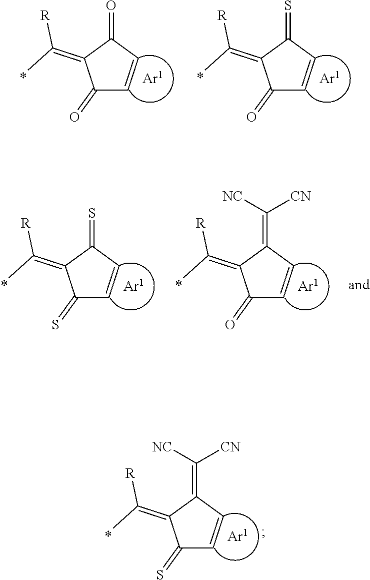

wherein each Ar¹ is independently an aromatic or heteroaromatic ring;
* represent the bond to L;
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
any two adjacent substituents R optionally join to form a fused ring.

In one embodiment, the divalent linker L promotes conjugation between linear ring system D and acceptor group A. In one embodiment, each divalent linker L is independently selected from the group consisting of an aryl ring, a heteroaryl ring, a cyclic alkene, a cyclic heteroalkene, and an alkyne. In one embodiment, the divalent linker L is not 1,2-cyclopentene. In one embodiment, one divalent linker L is not 1,2-cyclopentene. In one embodiment, one divalent linker L is not 1,2-cyclopentene. In one embodiment, no divalent linker L is not a single bond. In one embodiment, one divalent linker L is not a single bond.

In one embodiment, each L is independently selected from the group consisting of:

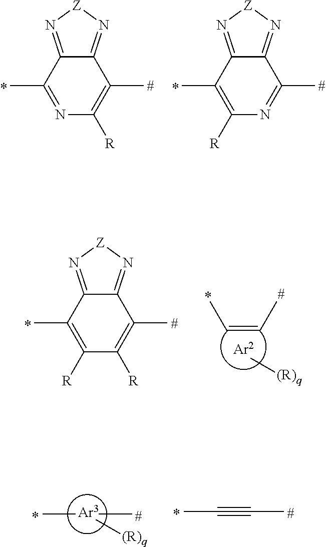

wherein * represents the bond to linear ring system D;
#represents the bond to A;
Z is NR, C(R)₂, O, S, Se, or Te;
Ar² and Ar³ are each an aryl or heteroaryl ring;
q is an integer between zero and the maximum possible substitution on Ar² or Ar³; and each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two adjacent R optionally join to form a ring.

In one embodiment, D comprises three rings D′, four rings D′, five rings D′, six rings D′, seven rings D′, eight rings D′, nine rings D′, ten rings D′, eleven rings D′, twelve rings D′, thirteen rings D′, fourteen rings D′, fifteen rings D′, sixteen rings D′, seventeen rings D′, eighteen rings D′, nineteen rings D′, twenty rings D′, twenty-one rings D′, twenty-two rings D′, twenty-three rings D′, twenty-four rings D′, or twenty-five rings D′.

In one embodiment, each ring D′ is independently selected from the group consisting of:

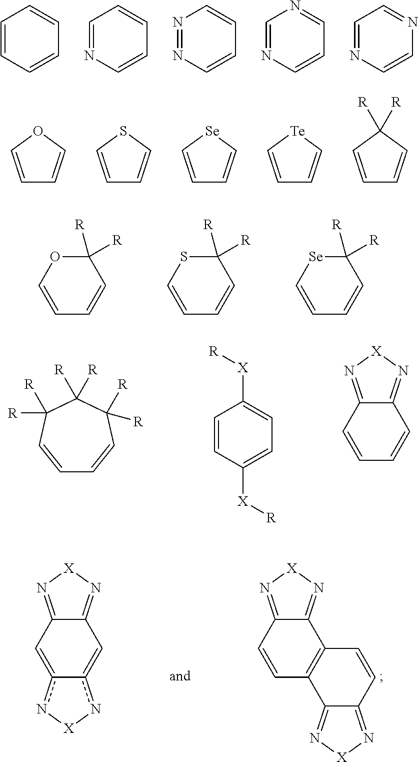

wherein each X is independently selected from the group consisting of oxygen, carbon, hydrogen, sulfur, selenium, and nitrogen; and
wherein each ring D′ is optionally substituted with one or more substituents R.

In one embodiment, each A^{r} is independently selected from the group consisting of:

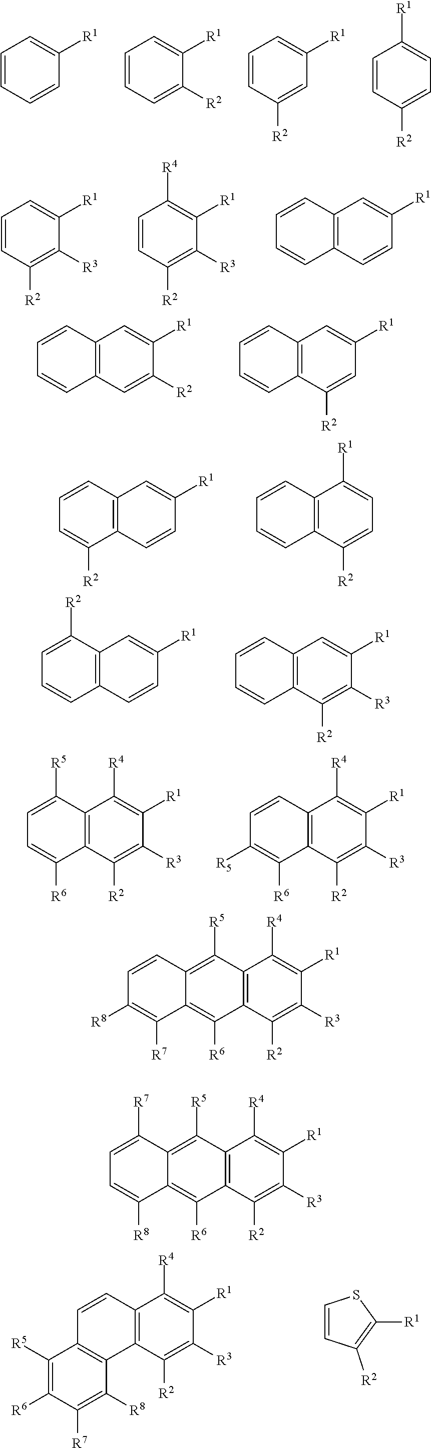

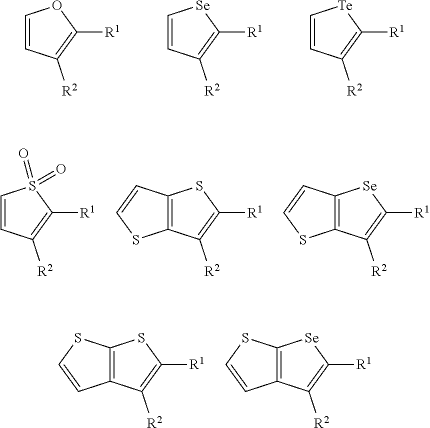

wherein R¹ to R⁸ each independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof.

In one embodiment, at least one occurrence of R¹ to R⁸ represents halogen.

In one embodiment, each of Ar² and Ar³ are independently selected from the group consisting of benzene, pyridine, pyrimidine, pyridazine, pyrazine, triazine, imidazole, pyrazole, pyrrole, oxazole, furan, thiophene, triazole, thiazole, naphthalene, quinoline, isoquinoline, quinazoline, benzofuran, benzoxazole, benzothiophene, benzothiazole, benzoselenophene, indene, indole, benzimidazole, carbazole, dibenzofuran, dibenzothiophene, quinoxaline, phthalazine, phenanthrene, phenanthridine, and fluorene.

In one embodiment, the compound is represented by Formula (II):

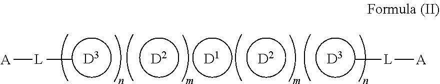

wherein:
m is an integer between 0 and 10;
n is an integer between 0 and 10;
D¹, each D², and each D³ is fused to or covalently bonded to an adjacent ring
D¹, D², and D³ are each independently a monocyclic ring or a polycyclic fused ring system, wherein the monocyclic ring or each ring of the polycyclic fused ring system is a 5-membered to 10-membered carbocyclic or heterocyclic ring; wherein each of D¹, D², and D³ is optionally further substituted with one or more substituents R; and
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two adjacent substituents R are optionally joined to form a fused ring.

In one embodiment, D¹ is represented by one of the following structures:

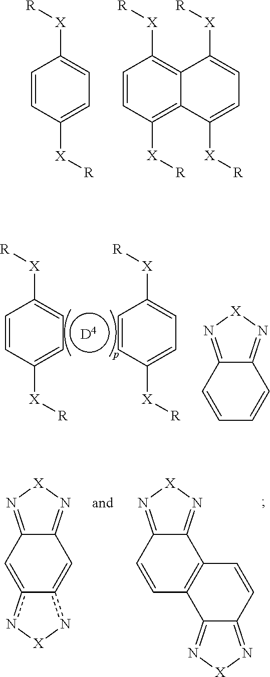

wherein:
each X is independently selected from the group consisting of oxygen, carbon, hydrogen, sulfur, selenium, and nitrogen;
p is an integer between 0 and 10;
wherein D⁴ is a monocyclic ring or a polycyclic fused ring system, wherein the monocyclic ring or each ring of the polycyclic fused ring system is a 5-membered to 10-membered carbocyclic or heterocyclic ring; wherein D⁴ is optionally further substituted with one or more substituents R; and wherein D¹ is optionally substituted with one or more substituents R.

In one embodiment, D² is selected from the group consisting of:

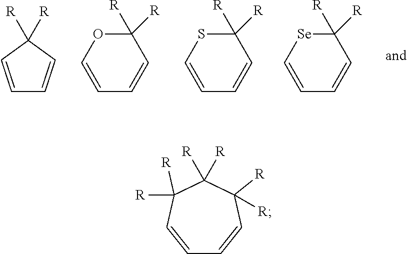

wherein each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two groups R optionally together form a ring.

In one embodiment, D³ is selected from the group consisting of:

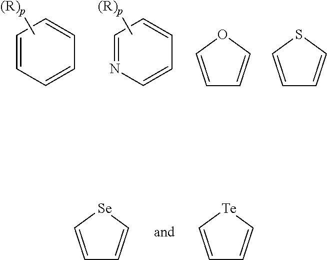

wherein p is an integer between 0 and 2; and
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two groups R optionally together form a ring.

In one embodiment, the compound is represented by Formula (III), Formula (IV), Formula (V), Formula (VI), Formula (VII), or Formula (VIII);

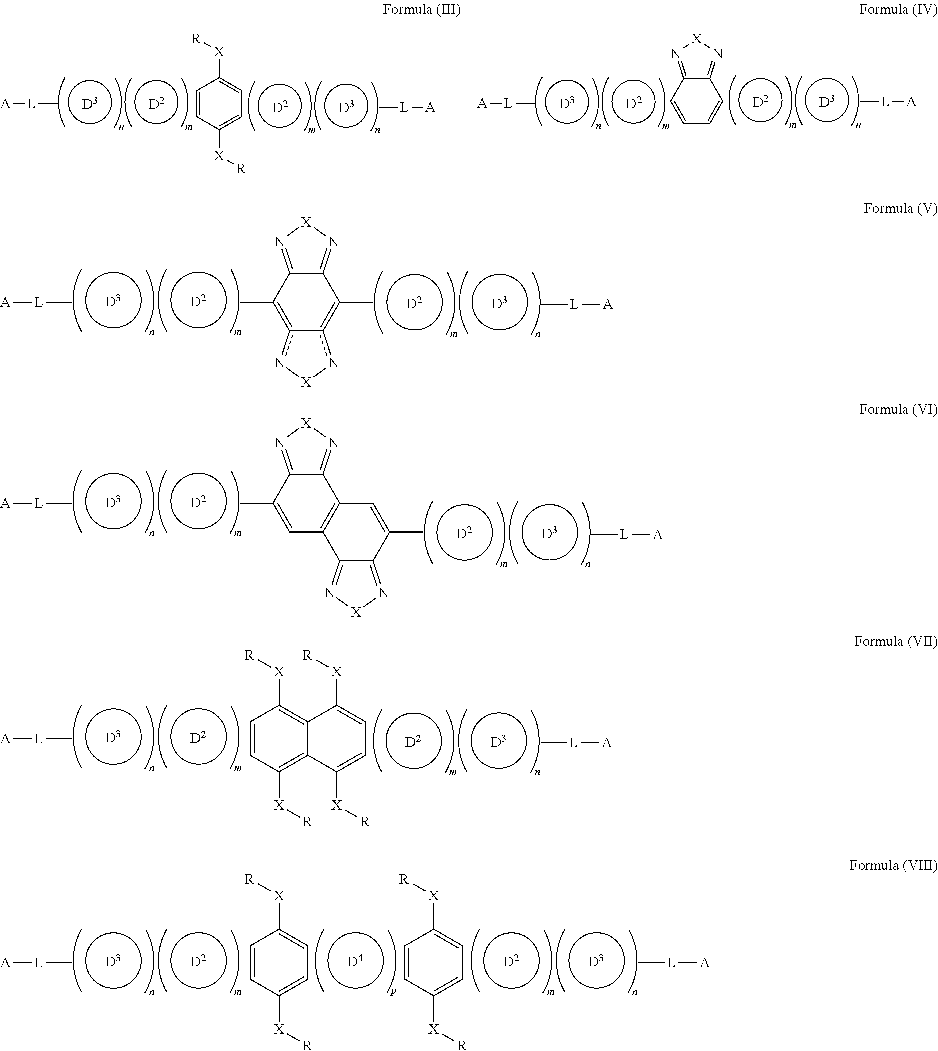

wherein
X is selected from the group consisting of oxygen, carbon, hydrogen, sulfur, selenium, and nitrogen.

In one embodiment, the compound is represented by one of the following formulas:

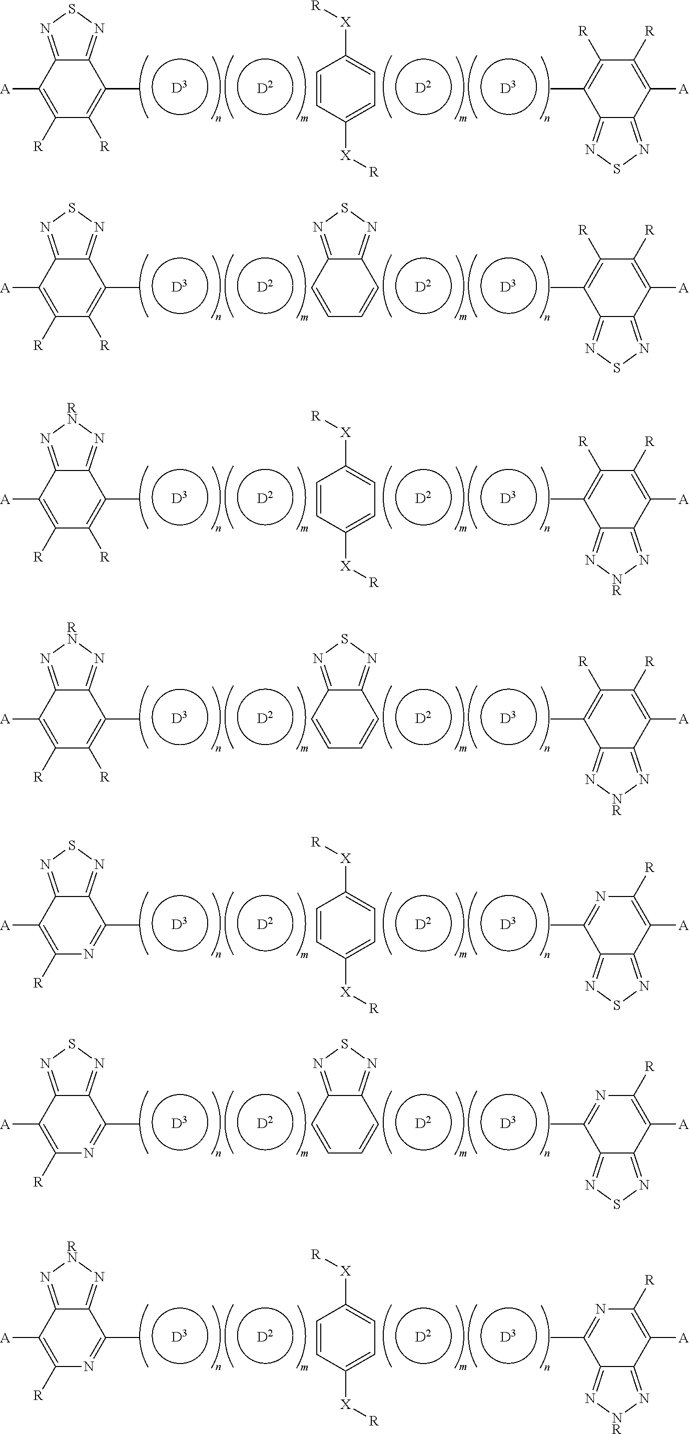

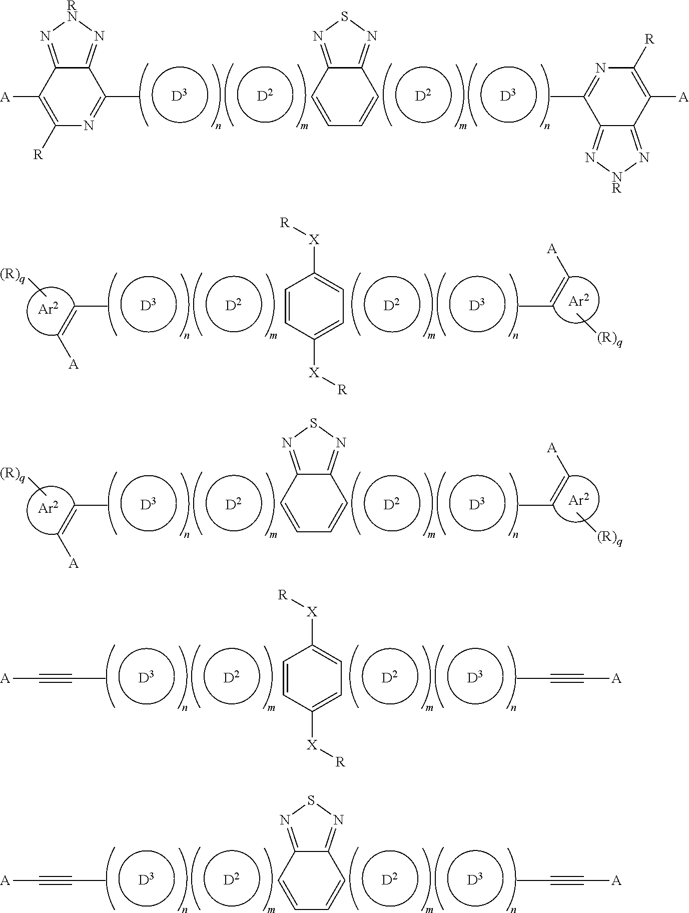

According to another aspect, a formulation comprising a compound described herein is also disclosed.

### Organic Photovoltaic Cells

In one aspect, the disclosure relates to an OPV device comprising a compound of the disclosure. In one embodiment, the OPV device includes an anode; a cathode; and an active material positioned between the anode and cathode, wherein the active material comprises an acceptor and a donor.

In one embodiment, the OPV device comprises a single-junction organic photovoltaic device. In one embodiment, the OPV device comprises two electrodes having an anode and a cathode in superposed relation, at least one donor composition, and at least one acceptor composition, wherein the donor-acceptor material or active layer is positioned between the two electrodes. In one embodiment, one or more intermediate layers may be positioned between the anode and the active layer. Additionally, or alternatively, one or more intermediate layers may be positioned between the active layer and cathode.

In one embodiment, the anode comprises a conducting oxide, thin metal layer, or conducting polymer. In one embodiment, the anode comprises a conductive metal oxide. Exemplary conductive metal oxides include, but are not limited to, indium tin oxide (ITO), tin oxide (TO), gallium indium tin oxide (GITO), zinc oxide (ZO), and zinc indium tin oxide (ZITO). In one embodiment, the anode comprises a metal layer. Exemplary metals for the metal layer include, but are not limited to, Ag, Au, Pd, Pt, Ti, V, Zn, Sn, Al, Co, Ni, Cu, Cr, and combinations thereof. In one embodiment, the metal layer comprises a thin metal layer. In one embodiment, the anode 102 comprises a conductive polymer. Exemplary conductive polymers include, but are not limited to, polyanaline (PANI), or 3,4-polyethyl-enedioxythiophene:polystyrenesulfonate (PEDOT:PSS). In one embodiment, thickness of the anode is between about 0.1-100 nm. In one embodiment, thickness of the anode is between about 1-10 nm. In one embodiment, thickness of the anode is between about 0.1-10 nm. In one embodiment, thickness of the anode is between about 10-100 nm. In one embodiment, anode comprises a transparent or semi-transparent conductive material.

In one embodiment, the cathode comprises a conducting oxide, a metal layer, or conducting polymer. Exemplary conducting oxide, metal layers, and conducting polymers are described elsewhere herein. In one embodiment, the cathode comprises a thin metal layer. In one embodiment, the cathode comprises a metal or metal alloy. In one embodiment, the cathode may comprise Ca, Al, Mg, Ti, W, Ag, Au, or another appropriate metal, or an alloy thereof. In one embodiment, the thickness of the cathode is between about 0.1-100 nm. In one embodiment, the thickness of the cathode is between about 1-10 nm. In one embodiment, the thickness of the cathode is between about 0.1-10 nm. In one embodiment, the thickness of the cathode is between about 10-100 nm. In one embodiment, cathode comprises a transparent or semi-transparent conductive material.

In one embodiment, the OPV device may comprise one or more charge collecting/transporting intermediate layers positioned between an electrode and the active region or layer. In one embodiment, the OPV device comprises one or more intermediate layers. In one embodiment, the intermediate layer comprises a metal oxide. Exemplary metal oxides include, but are not limited to, MoO₃, MoOx, V₂O₅, ZnO, and TiO₂. In one embodiment, the first intermediate layer has the same composition as the second intermediate layer. In one embodiment, the first intermediate layer and the second intermediate layer have different compositions. In one embodiment, the thickness of the intermediate layers are each independently between about 0.1-100 nm. In one embodiment, the thickness of the intermediate layers are each independently between about 1-10 nm. In one embodiment, the thickness of the intermediate layers are each independently between about 0.1-10 nm. In one embodiment, the thickness of the intermediate layers are each independently between about 10-100 nm.

In one embodiment, the OPV device comprises various layers of a tandem or multi-junction photovoltaic device. In one embodiment, the OPV device comprises two electrodes having an anode and a 204 in superposed relation, at least one donor composition, and at least one acceptor composition positioned within a plurality of active layers or regions between the two electrodes. Additional active layers or regions are also possible. In one embodiment, the anode and the cathode each independently comprise a conducting oxide, thin metal layer, or conducting polymer. Exemplary conducting oxides, metal layers, and conducting polymers are described elsewhere herein.

In one embodiment, the OPV device comprises one or more intermediate layers positioned between the anode and a first active layer. Additionally, or alternatively, at least one intermediate layer may be positioned between the second active layer and cathode. In one embodiment, the OPV device comprises one or more intermediate layers positioned between the first active layer and the second active layer. In one embodiment, the OPV device comprises a first intermediate layer. In one embodiment, the OPV device comprises a second intermediate layer. In one embodiment, the OPV device comprises a third intermediate layer. In one embodiment, the OPV device comprises both first and second intermediate layers. In one embodiment, the OPV device comprises both first and third intermediate layers. In one embodiment, the OPV device comprises both second and third intermediate layers. In one embodiment, the OPV device comprises first, second, and third intermediate layers. In one embodiment, the first, second, and/or third intermediate layer comprises a metal oxide. Exemplary metal oxides are described elsewhere herein.

### EXPERIMENTAL EXAMPLES

The highest efficiency organic photovoltaic (OPV) cells to date have achieved efficiencies approaching 20%. In addition, OPVs have shown extraordinary stabilities of hundreds if not thousands of years. However, the highest efficiency solar cells (although not necessarily the longest lived) comprise polymer donors and solution deposited non-fullerene acceptors, which compares with only 11% power conversion efficiency (PCE) demonstrated for all-vacuum-deposited OPVs. This is in contrast to the extraordinarily successful, >$50 billion organic LED (OLED) industry which is completely dominated by vacuum deposited materials and structures. A primary reason for the adoption of vacuum-deposited materials in OLEDs is due to the ability to purify the source materials to achieve high performance, reliability and reproducibility. To take full advantage of the manufacturing infrastructure and depth of know-how available to OLEDs, it is imperative that OPVs based on vapor-deposited materials eventually achieve efficiencies comparable to, or even exceeding those employing solution-processed materials. While high efficiency vacuum deposition-compatible donors have been achieved, similarly deposited acceptors have not yet been developed. Here, we disclosure a series of vacuum-deposited NFA materials with absorption extend to 1000 nm and great chemical stability. This is critical to enhancing the reproducibility as well as reliability of vacuum-deposited OPVs.

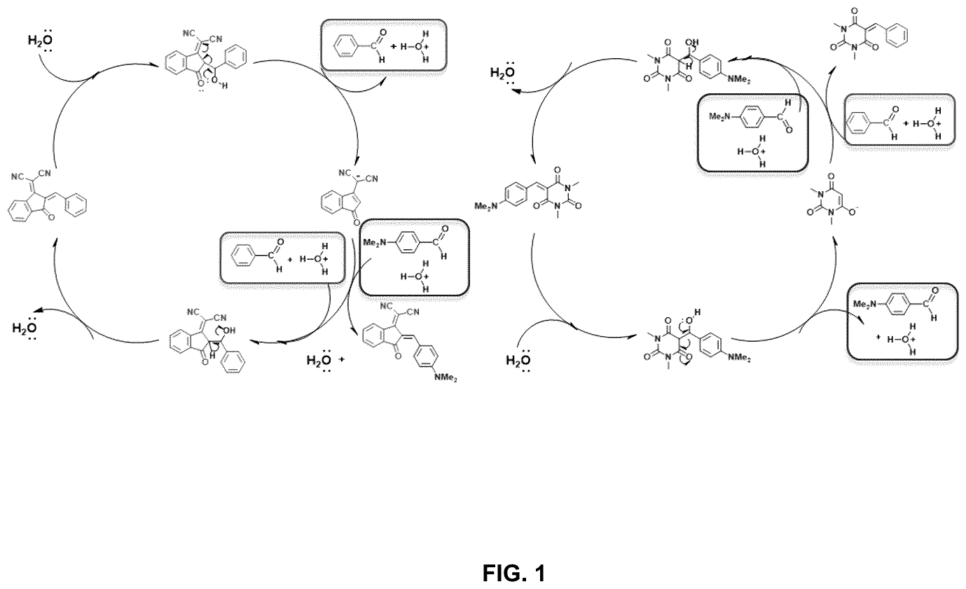

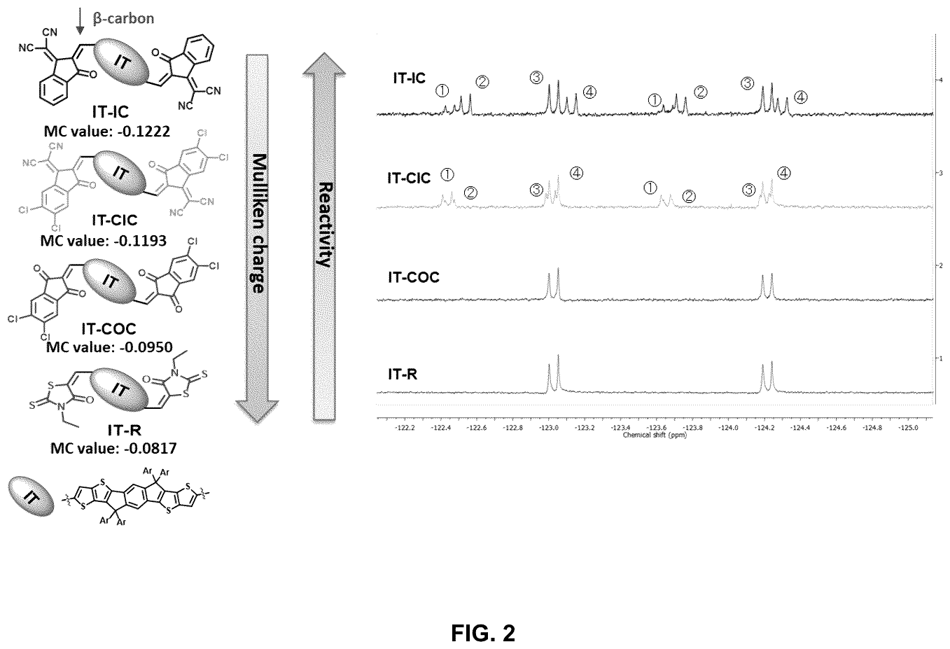

In a recent study, it was found that acceptor-donor-acceptor (a-d-a) type of non-fullerene acceptors (NFAs) used in high efficiency OPV cells undergo dissociative reactions during thermal evaporation that create a plethora of reaction products (FIG. 1). The vinyl groups linking the D and A moieties can be dissociated, and the reactivity of the dissociation is strongly correlated with the polarizability of C═C double bonds (FIG. 2). Here is disclosed a series of vacuum-deposited NFA materials with absorption extend to 1000 nm and great chemical stability. This is critical to enhancing the reproducibility as well as reliability of vacuum-deposited OPVs.

The first strategy is to distribute the charge localized at the C in the vinyl linkage by insertion of a second, conjugated acceptor such as a benzothiadiazole.

An alternative strategy also proposed for this program is to use double bond cyclization by bridging the acceptor moiety to the thiophene chain. This may slightly blue-shift the absorption which can be counteracted by extending the thiophene backbone.

One final strategy is to replace the vinyl with a triple-bonded alkyne group. All three approaches distribute the electron density localized on the b-carbon site on the vinyl linkage over several bonds, presumably making the entire structure sufficiently stable to allow for thermal evaporation while not tampering with the basic a-d-a structure that has led to such high efficiency OPVs over the last several years.

It is understood that the various embodiments described herein are by way of example only, and are not intended to limit the scope of the disclosure. For example, many of the materials and structures described herein may be substituted with other materials and structures without deviating from the spirit of the disclosure. The present compounds as disclosed may therefore include variations from the particular examples and preferred embodiments described herein, as will be apparent to one of skill in the art. It is understood that various theories as to why the embodiments work are not intended to be limiting.

We claim:

1. A compound having a structure represented by Formula (I): 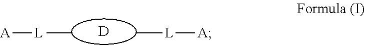
wherein:
D represents a linear ring system of between three and twenty-five rings D′; wherein each ring D′ is independently a monocyclic ring or a polycyclic fused ring system; and wherein each ring D′ is a 5-membered to 10-membered carbocyclic or heterocyclic ring; wherein each ring D′ is fused to an adjacent ring D′ or bonded to an adjacent ring D′ via a single bond; and wherein each ring D′ is optionally substituted with one or more substituents R;
each L is independently a divalent linker covalently bonded to a terminal ring D′ and to A; and
each A is independently an acceptor group selected from the group consisting of: 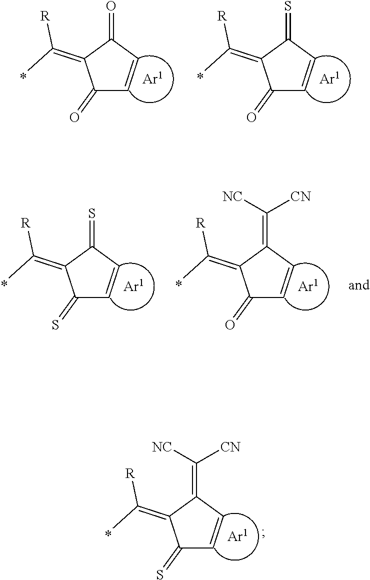
wherein each Ar¹ is independently an aromatic or heteroaromatic ring;
* represent the bond to L;
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
any two adjacent substituents R optionally join to form a fused ring.

2. The compound of claim 1, wherein each L is independently selected from the group consisting of an aryl ring, a heteroaryl ring, a cyclic alkene, a cyclic heteroalkene, and an alkyne; wherein each L is optionally substituted with one or more substituents R.

3. The compound of claim 1, wherein each L is independently selected from the group consisting of: 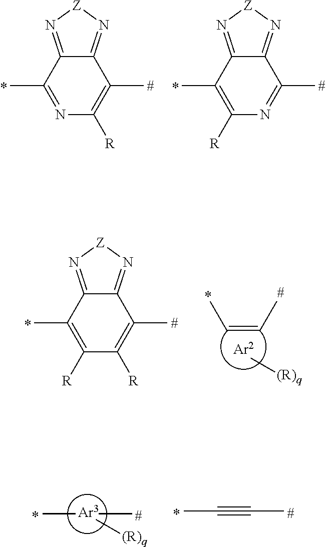
wherein * represents the bond to linear ring system D;
#represents the bond to A;
Z is NR, C(R)₂, O, S, Se, or Te;
Ar² and Ar³ are each an aryl or heteroaryl ring;
q is an integer between zero and the maximum possible substitution on Ar² or Ar³; and
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two adjacent R optionally join to form a ring.

4. The compound of claim 1, wherein D comprises six rings D′, seven rings D′, or eight rings D′.

5. The compound of claim 1, wherein each ring D′ is independently selected from the group consisting of: 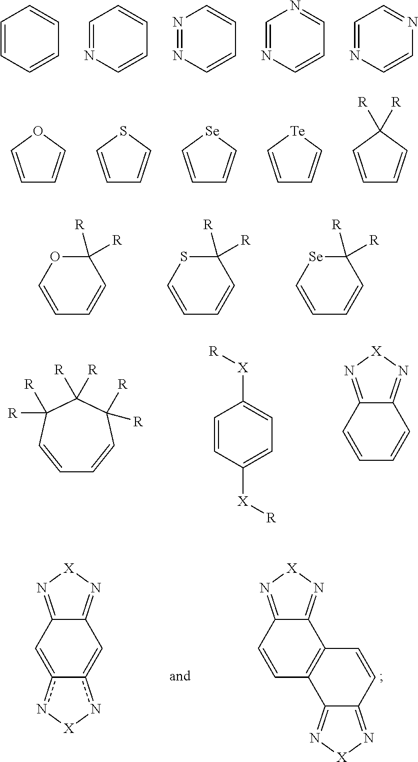
wherein each X is independently selected from the group consisting of oxygen, carbon, hydrogen, sulfur, selenium, and nitrogen; and
wherein each ring D′ is optionally substituted with one or more substituents R.

6. The compound of claim 1, wherein each Ar¹ is independently selected from the group consisting of: 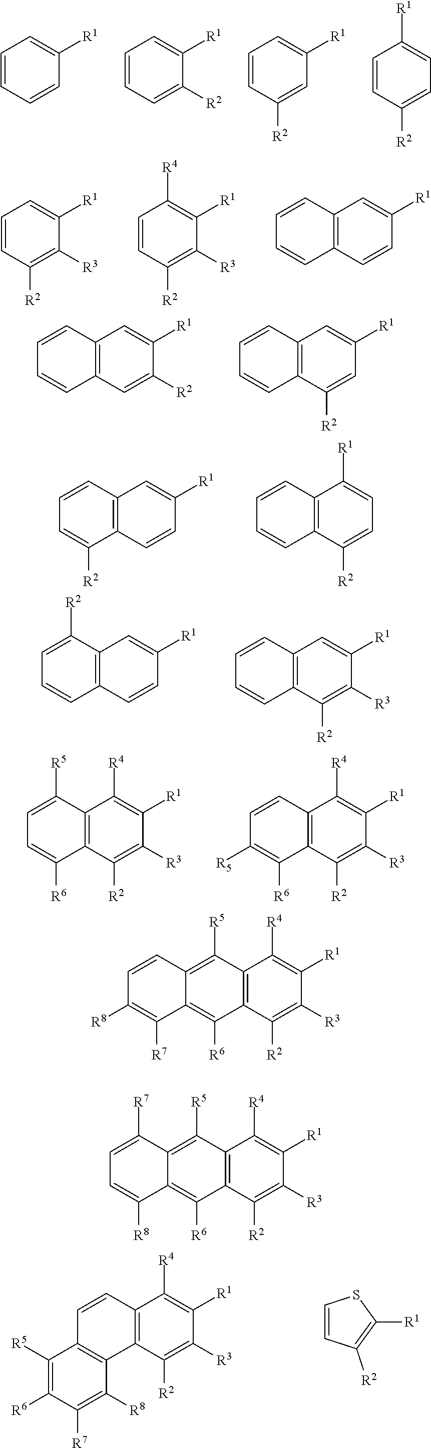 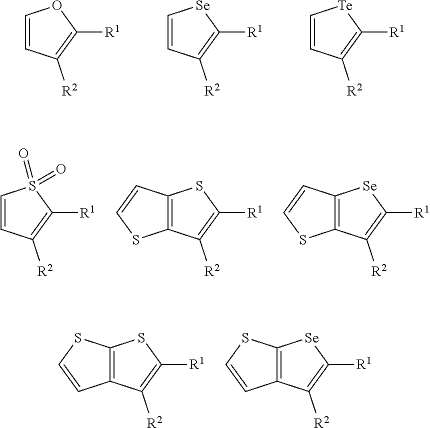
wherein R¹ to R⁸ each independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof.

7. The compound of claim 6, wherein at least one of R¹ to R⁸ represents halogen.

8. The compound of claim 1, wherein the compound is represented by Formula (II): 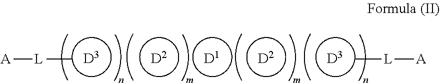
wherein:
m is an integer between 0 and 10;
n is an integer between 0 and 10;
D¹, each D², and each D³ is fused to or covalently bonded to an adjacent ring D¹, D², and D³ are each independently a monocyclic ring or a polycyclic fused ring system, wherein the monocyclic ring or each ring of the polycyclic fused ring system is a 5-membered to 10-membered carbocyclic or heterocyclic ring; wherein each of D¹, D², and D³ is optionally further substituted with one or more substituents R; and
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two adjacent substituents R are optionally joined to form a fused ring.

9. The compound of claim 7, wherein D¹ is represented by one of the following structures: 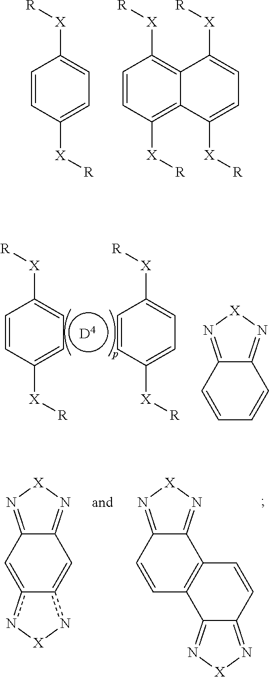
wherein:
each X is independently selected from the group consisting of oxygen, carbon, hydrogen, sulfur, selenium, and nitrogen;
p is an integer between 0 and 10;
wherein D⁴ is a monocyclic ring or a polycyclic fused ring system, wherein the monocyclic ring or each ring of the polycyclic fused ring system is a 5-membered to 10-membered carbocyclic or heterocyclic ring; wherein D⁴ is optionally further substituted with one or more substituents R; and
wherein D¹ is optionally substituted with one or more substituents R.

10. The compound of claim 8, wherein D² is selected from the group consisting of: 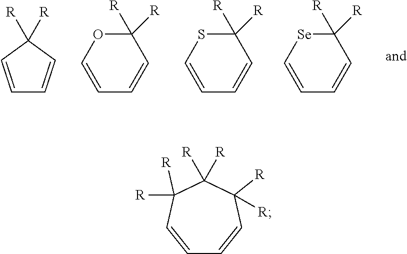
wherein each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two groups R optionally together form a ring.

11. The compound of claim 8, wherein D³ is selected from the group consisting of: 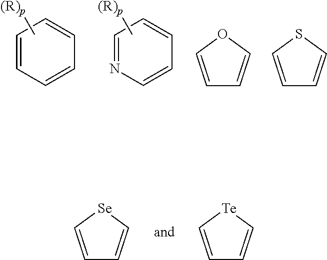
wherein p is an integer between 0 and 2; and
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two groups R optionally together form a ring.

12. The compound of claim 8, wherein the compound is represented by Formula (III), Formula (IV), Formula (V), Formula (VI), Formula (VII), or Formula (VIII); 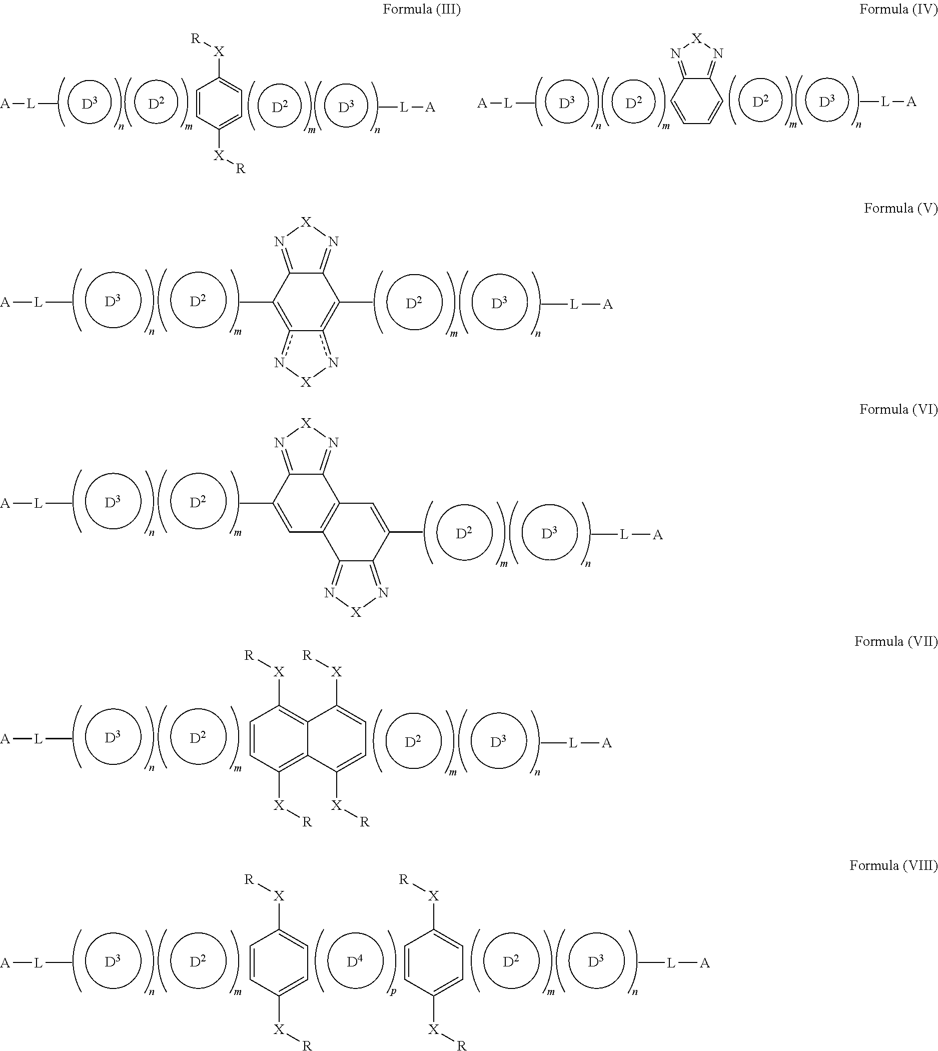
wherein
X is selected from the group consisting of oxygen, carbon, hydrogen, sulfur, selenium, and nitrogen.

13. The compound of claim 8, wherein the compound is represented by one of the following formulas:

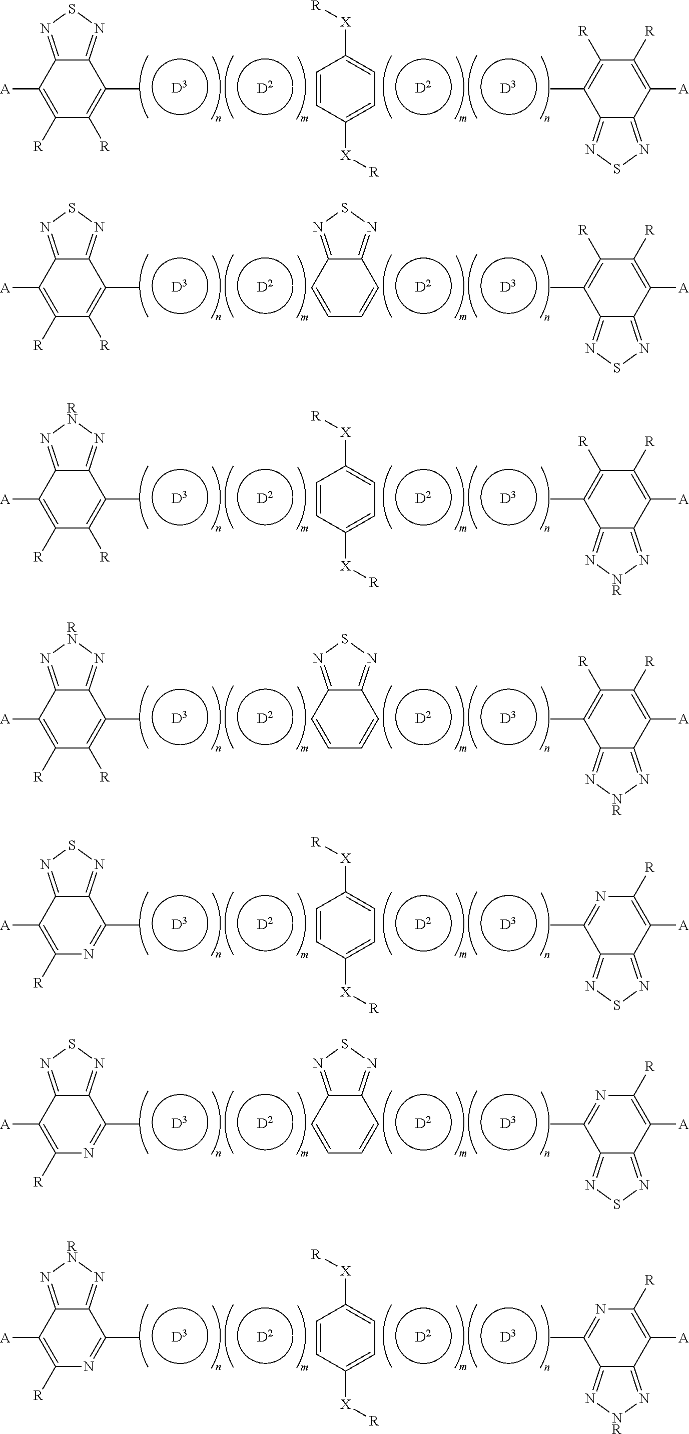

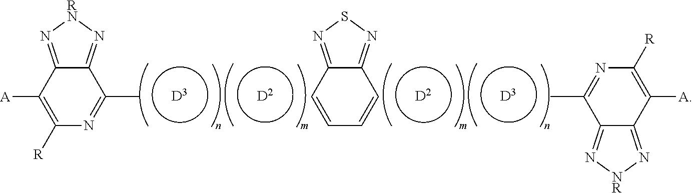

14. The compound of claim 8, wherein the compound is represented by the one of the following formulas:

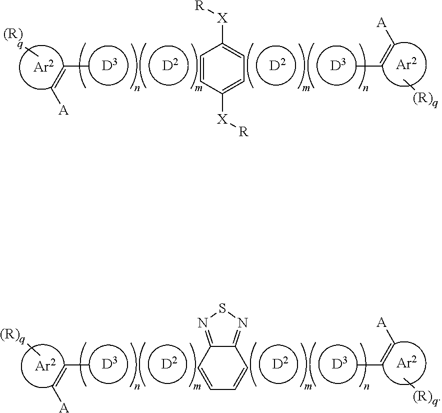

15. The compound of claim 8, wherein the compound is represented by one of the following formulas:

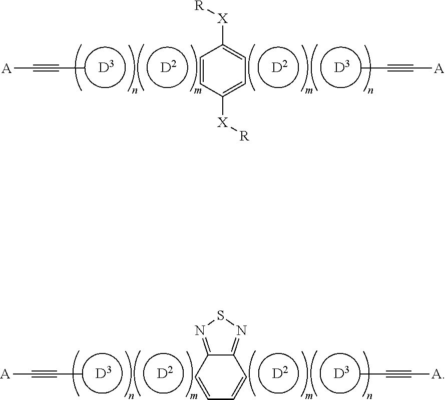

16. An optoelectronic device comprising a compound represented by Formula (I): 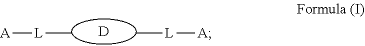
wherein:
D represents a linear ring system of between three and twenty-five rings D′; wherein each ring D′ is independently a monocyclic ring or a polycyclic fused ring system; and wherein each ring D′ is a 5-membered to 10-membered carbocyclic or heterocyclic ring; wherein each ring D′ is fused to an adjacent ring D′ or bonded to an adjacent ring D′ via a single bond; and wherein each ring D′ is optionally substituted with one or more substituents R;
each L is independently a divalent linker covalently bonded to a terminal ring D′ and to A; and
each A is independently an acceptor group selected from the group consisting of: 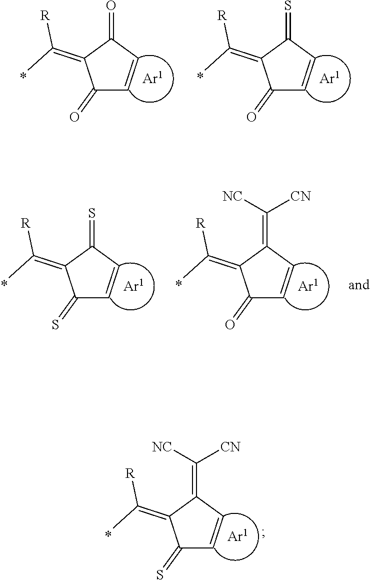
wherein each Ar¹ is independently an aromatic or heteroaromatic ring;
* represent the bond to L;
each occurrence of R independently represents hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
any two adjacent substituents R optionally join to form a fused ring.

17. The optoelectronic device of claim 16, wherein the optoelectronic device is selected from the group consisting of an organic light-emitting device (OLED), organic phototransistor, organic photovoltaic cell, and organic photodetector.

18. The optoelectronic device of claim 16, wherein the optoelectronic device is a photovoltaic cell.

19. A consumer product comprising the optoelectronic device of claim 16.

20. A formulation comprising the compound of claim 1.
